(19) United States
(12) Patent Application Publication
(10) Pub. No.: US 2026/0061203 A1
(43) Pub. Date: Mar. 5, 2026

(54) CONFIGURABLE SINGLE ACTUATION MECHANISM PROGRAMMING REMOTE FOR MEDICAL DEVICES

(71) Applicant: Medtronic, Inc., Minneapolis, MN (US)

(72) Inventors: Andrew T. Fried, St. Paul, MN (US); Jacob P. Komarek, Fridley, MN (US); Jacob D. Chatterton, Roseville, MN (US); Joel A. Anderson, Brooklyn Park, MN (US); Bernard P. Bechara, Hopkins, MN (US); Arthur K. Lai, Minnetonka, MN (US); Jermaine O. Stewart, Maple Grove, MN (US)

(21) Appl. No.: 19/314,807

(22) Filed: Aug. 29, 2025

Related U.S. Application Data
(60) Provisional application No. 63/690,185, filed on Sep. 3, 2024.

(51) Int. Cl. A61N 1/372; A61N 1/36

(52) U.S. Cl. CPC A61N 1/37247; A61N 1/37264; A61N 1/36128

(57) ABSTRACT

A medical device system includes a handheld patient remote, the handheld patient programming remote having a configurable single actuation mechanism; and processing circuitry configured to: during a programming phase of a medical device, receive an indication of a type of the medical device to be in communication with the handheld patient programming remote; during the programming phase, receive user input indicative of a user preference; based on the indication of the type of medical device and the user preference, determine a function to be performed with the single actuation mechanism of the handheld patient remote; and configure the single actuation mechanism to perform, in response to patient action, the determined operation.

This application claims the benefit of U.S. Provisional Patent Application No. 63/690,185, filed 3 Sep. 2024, the entire contents of which is incorporated herein by reference.

### TECHNICAL FIELD

This disclosure generally relates to medical devices, and more particularly, to a medical device programming system.

### BACKGROUND

A variety of medical devices use and/or include electrodes to facilitate sensing physiological signals and/or to provide stimulation. Medical devices may be external or implanted and may be used to deliver electrical stimulation therapy to various tissue sites of a patient to treat a variety of symptoms or conditions such as chronic pain, tremor, Parkinson's disease, other movement disorders, epilepsy, tachyarrhythmia, urinary or fecal incontinence, sexual dysfunction, obesity, or gastroparesis. A medical device may deliver electrical stimulation therapy via one or more leads that include electrodes located proximate to target locations. Hence, electrical stimulation may be used in different therapeutic applications, such as deep brain stimulation (DBS), cardiac rhythm management, spinal cord stimulation (SCS), pelvic stimulation, e.g., sacral neuromodulation and tibial neuromodulation, gastric stimulation, or peripheral nerve field stimulation (PFNS). Some medical devices include telemetry circuitry to communicate with other devices, such as programming remotes.

### SUMMARY

In general, the disclosure describes devices, systems, and techniques for configuring a handheld patient programming remote with a configurable limited user interface (e.g., configurable single actuation mechanism) to establish a communication link with a medical device, e.g., via Bluetooth® or Bluetooth® Low Energy (BLE), to power the medical device on and/or off, e.g., to power therapy on and/or off or to switch between a relatively lower power and a relatively higher power, or to adjust a therapy delivery of the device, e.g., to disable and/or enable a scheduled therapy session. In one or more examples, the single actuation mechanism may be configured to perform one of the above example functions, and then reconfigured to perform a different function. In some examples, the single actuation mechanism may be configured to perform different functions for different types of medical devices.

The handheld patient programmer includes a limited number of programming functions. In some examples, the handheld patient programming remote may be capable of communicating with any of a plurality of types of medical devices, e.g., a tibial neuromodulation (TNM) device, a spinal cord stimulation (SCS) device, and a sacral neuromodulation device. During a clinician programming session, during manufacturing, or during distribution, the handheld patient programming remote may receive an indication of which type of medical device of the plurality of types of medical devices the handheld patient programming remote will be configured to communicate with. In some examples, the indication may be a selection of one of a plurality of business logics corresponding to the type of medical device.

In addition to the indication of the type of medical device that the handheld patient programming remote will be configured to interact with, the handheld patient programming remote may additionally receive user input, e.g., clinician and/or patient input, indicative of a preferred functionality of the handheld patient programming remote. For example, if the indicated type of medical device is a tibial stimulation device, in some patients, stimulation may be configured to be delivered on demand, and in other patients, stimulation may be configured to be delivered according to a scheduled therapy program. Patients who receive on demand stimulation may want to turn the tibial stimulation device on and off. Patients who receive stimulation according to a scheduled therapy program may want to cancel a scheduled therapy session from time to time. The clinician and/or patient may provide user input indicating a preferred functionality the handheld patient programming remote, e.g., an on/off functionality or a cancel scheduled therapy functionality.

The handheld patient programming remote may have a single function and is sized to fit in a pocket in a patient's clothing. For example, the handheld patient programming remote may be the approximate size of a key fob for an automobile keyless entry system. In some examples, the handheld patient programming remote may include a keychain holder and may be configured to be placed on a keychain for patient convenience. For example, the keychain holder and size may improve portability and prevent the patient from misplacing the handheld patient programming remote.

In one example, a medical device system includes a handheld patient remote, the handheld patient programming remote having a configurable single actuation mechanism; and processing circuitry configured to: during a programming phase of a medical device, receive an indication of a type of the medical device to be in communication with the handheld patient programming remote; during the programming phase, receive user input indicative of a user preference; based on the indication of the type of medical device and the user preference, determine a function to be performed with the single actuation mechanism of the handheld patient remote; and configure the single actuation mechanism to perform, in response to patient action, the determined operation.

In another example, a method includes receiving, by processing circuitry of a medical device system and during a programming phase of a medical device, an indication of a type of the medical device to be in communication with a handheld patient programming remote, the handheld patient programming remote having a configurable single actuation mechanism; receiving, by the processing circuitry and during the programming phase, user input indicative of a user preference; determining, by the processing circuitry and based on the indication of the type of medical device and the user preference, a function to be performed with the single actuation mechanism of the handheld patient remote; and configuring, by the processing circuitry, the single actuation mechanism to perform, in response to patient action, the determined operation.

In another example, a non-transitory computer-readable medium stores instructions that when executed cause processing circuitry to: during a programming phase of a medical device, receive an indication of a type of the medical device to be in communication with a handheld patient programming remote, the handheld patient programming remote having a configurable single actuation mechanism; during the programming phase, receive user input indicative of a user preference; based on the indication of the type of medical device and the user preference, determine a function to be performed with the single actuation mechanism of the handheld patient remote; and configure the single actuation mechanism to perform, in response to patient action, the determined operation.

The details of one or more examples of the techniques of this disclosure are set forth in the accompanying drawings and the description below. Other features, objects, and advantages of the techniques will be apparent from the description and drawings, and from the claims.

### BRIEF DESCRIPTION OF DRAWINGS

FIG. 1 is a conceptual diagram illustrating an example system including a tibial stimulation device, a handheld patient programming remote, and a clinician programmer according to one or more techniques of this disclosure.

FIG. 2 is a conceptual diagram illustrating an example system including a sacral neuromodulation device, a handheld patient programming remote, and a clinician programmer according to one or more techniques of this disclosure.

FIG. 3 is a conceptual diagram illustrating an example system in which a handheld patient programming remote may be used to communicate with an implantable medical device (IMD) according to one or more techniques of this disclosure.

FIG. 4 is a conceptual diagram illustrating an example embodiment of a handheld patient programming device, according to one or more techniques of this disclosure.

FIG. 5 is a block diagram illustrating an example configuration of a handheld patient programming remote according to one or more techniques of this disclosure.

FIG. 6 is a flow chart illustrating an example operation for configuring a single actuation mechanism of a handheld patient programming remote according to one or more techniques of this disclosure.

FIG. 7 is a flow chart illustrating an example operation for determining a type of medical device to be in communication with a handheld patient programming remote according to one or more techniques of this disclosure.

FIG. 8 is a flow chart illustrating an example operation for a handheld patient programming remote performing a power on and off function according to one or more techniques of this disclosure.

FIG. 9 is a flow chart illustrating an example operation for a handheld patient programming remote performing a wake up function according to one or more techniques of this disclosure.

FIG. 10 is a flow chart illustrating an example operation for a handheld patient programming remote performing a switching between programs function according to one or more techniques of this disclosure.

FIG. 11 is a flow chart illustrating an example operation for a handheld patient programming remote performing a wake up function according to one or more techniques of this disclosure.

### DETAILED DESCRIPTION

Medical devices may be external or implanted and may be used to deliver electrical stimulation therapy to patients via various tissue sites to treat a variety of symptoms or conditions urinary or fecal incontinence, urinary frequency, or urinary retention. A medical device may deliver peripheral nerve stimulation (PNS), including tibial nerve stimulation.

The medical device may be part of a medical device system including a handheld patient programming remote, a clinician programmer, and, in some examples, a user device, such as a patient smartphone, with an application. In some examples, the patient may perform patient programming using the handheld patient programming remote. However, in many cases, current external programming devices include features such as a liquid crystal display (LCD), haptics, audio, touch screen, front and side control buttons, and multiple layers of software. In some examples, these complex external programming devices can overwhelm and confuse patients. Current external programming devices may be more complex than the patient needs or wants. The patient may prefer a relatively small, discreet, and simple external programming device comprising relatively fewer buttons, e.g., an on/off button.

This disclosure is directed to a handheld patient programming remote with a configurable single actuation mechanism, e.g., a button. The handheld patient programming remote may run on (e.g., be powered by) a coin cell battery and configured to perform a single function when the button is pressed, such as powering a medical device, e.g., an implantable medical device (IMD), on and off, e.g., powering therapy of the IMD on and off, disabling and enabling scheduled therapies, waking up the IMD, or changing from one IMD behavior to another. The single button remote may include a button and one or more light emitting diodes (LEDs) disposed on the surface of the remote. The LED(s) may be on the button, may surround the button, or may be adjacent to the button. The function that the single actuation mechanism performs may be configurable.

The handheld patient programming remote can include a proximal antenna and proximal transmit and receive capabilities. In some cases, when the button is pressed by a user, e.g., a patient, the handheld patient programming remote may send wake up packages to cause the IMD to transition from a low power state to a high power state in which the handheld patient programming remote starts advertising at a relatively high rate, e.g., 1 Hertz, and allow the IMD to remain in a relatively low power state when a connection is unnecessary. In some examples, the handheld patient programming remote may listen for the advertising signals. In some examples, the handheld patient programming remote may connect to the IMD. In examples in which the handheld patient programming remote connects to the IMD, the single function of the button may switch from the wake up function to another function, such as powering the IMD therapy on and off. In some examples, the IMD may connect to other devices, such as a clinician programmer and/or a patient electronic device. In some examples, the handheld patient programming remote may stop sending wake up packages after a threshold period of time has elapsed. In some examples, the handheld patient programming remote may stop sending wake up packages in response to determining the IMD has increased an advertising packet rate. In some examples, when a connection is established, the one or more LEDs may change, e.g., from red to green or from blinking to solid. In some examples, the patient electronic device may control the patient programming remote to perform the same function as the button press and instruct the patient to place the remote over the IMD.

In some examples, when the button is pressed by the user, the handheld patient programming remote may power the IMD therapy on or off or may enable or disable a scheduled therapy. In some examples, the one or more LEDs may change color, e.g., from red to green or white to blue, when therapy is switched from off to on or from disabled to enabled.

The handheld patient programming remote may be approximately circular in shape, with a diameter within about 5 centimeters (cm) and a profile thickness of less than 1 cm. However, various sizes and shapes are also possible. For example, the handheld patient programming remote may be approximately rectangular, e.g., 5.1 cm by 4.1 cm, in shape, with a profile thickness of 1 cm or less. The handheld patient programming remote may include rounded corners and/or edges. In some examples, a housing of the single button remote may include an attachment portion, e.g., “a keychain holder,” allowing the patient to loop the single button remote onto a keychain, bag, lanyard, etc. In some examples, due to the small size of the device, the patient may be able to keep the single button remote in another discreet location, such as in a wallet, a purse, a backpack, on a phone, tucked in a sock, or attached to the laces of a pair of shoes. Due to the relatively small size of the handheld patient programming remote, the techniques of this disclosure may provide a discreet way to communicate with an implanted medical device.

The handheld patient programming remote may have a single function. In this way, the handheld patient programming remote may provide a simple way for the patient to interact with the IMD. In some examples, the patient may not need to interact with the IMD beyond powering the IMD therapy on and off, waking up the IMD, or enabling and disabling therapy, making additional functions unnecessary for the handheld patient programming remote. By limiting functionality of the handheld patient programming remote to a single function, the techniques of this disclosure may simplify patient interactions with the IMD, which may make the patient feel more comfortable with the IMD.

The handheld patient programming remote may be powered by a coin cell battery. In some examples, by limiting the functionality of the handheld patient programming remote, the techniques of this disclosure may advantageously increase a battery life of the remote, thereby increasing remote longevity. Increasing remote longevity may allow a patient to operate the handheld patient programming remote for a relatively longer period of time, which may reduce patient burden. For example, some watches last for 10 years running on a single primary cell battery. Similar performance may be possible with such a remote when interactions with the IMD are relatively infrequent and only triggered by a button press, which switches the handheld patient programming remote from a low power state to a high power state to facilitate communication with the IMD. Patients may also value the discreetness of such a design when said patients may use the handheld patient programming remote in public.

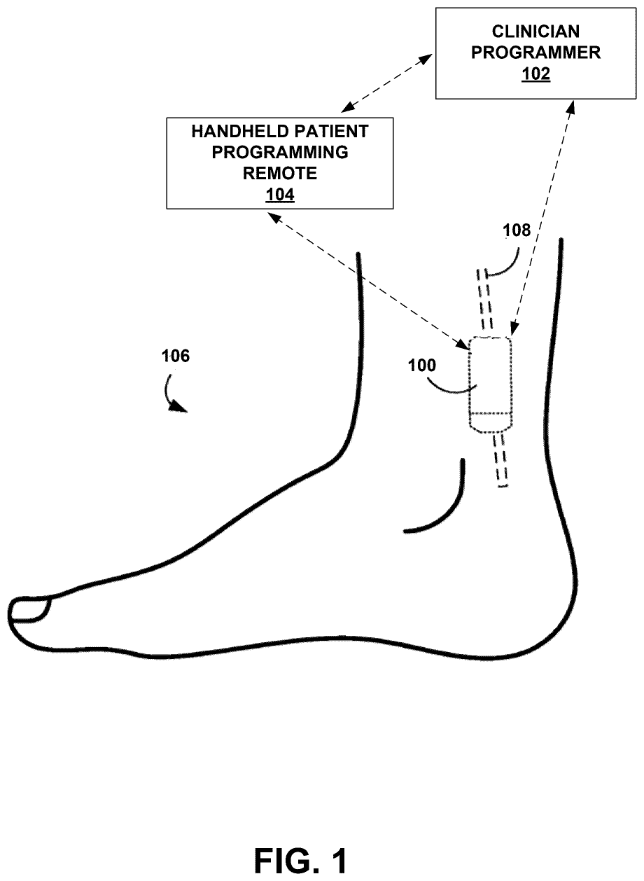

FIG. 1 is a conceptual diagram illustrating an example system including a tibial stimulation device 100, a handheld patient programming remote 104, and a clinician programmer 102 according to one or more techniques of this disclosure. Tibial stimulation device 100 may also be referred to as an implantable medical device (IMD) 100. For case of description, the examples are described with respect to IMD 100, but the techniques should not be considered limited to implantable medical devices and may be applicable to medical devices generally, e.g., external neurostimulators.

Clinician programmer 102 and handheld patient programming remote 104 may be configured to control the operation of IMD 100. In some examples, a clinician may use clinician programmer 102 to program IMD 100 periodically, e.g., during clinic visits or remotely in response to a patient request or other stimulus. In some examples, clinician programmer 102 may communicate with IMD 100 to download recorded data, to adjust one or more therapy settings, etc. Clinician programmer 102 may include a housing to enclose operational components such as a processor, memory, user interface, telemetry circuitry, and power source. Examples of programmer 104 include a tablet computer, laptop, a smartphone, a dedicated handheld device, or other similar computing devices. In some examples, a patient may use handheld patient programming remote 104 to power IMD 100 on and off or to enable or disable a scheduled therapy. Handheld patient programming remote 104 may comprise a dedicated handheld device with a single actuation mechanism, e.g., a button and/or a sensor configured to sense a particular motion or placement of handheld patient programming remote 104. In some examples, a user device, such as a patient smartphone with an application (not depicted), may additionally be configured to control the operation of IMD 100.

In some examples, clinician programmer 102 may have relatively greater control over the operation of IMD 100 than handheld patient programming remote 104 or the user device. For example, the clinician may set one or more stimulation amplitude limits and/or set a therapy schedule using clinician programmer 102, and the patient may be able to adjust therapy within the limits and/or disable a scheduled therapy via the user device. Additionally, the clinician, using clinician programmer 102, may configure handheld patient programming remote 104 by configuring a function to be performed by actuating the single actuation mechanism, e.g., pressing the button, of handheld patient programming remote 104. The clinician programmer 102 may also have the capability to communicate user preferences to the IMD that the remote 104 can later use in determining which behavior(s) to perform with the IMD. Alternatively, the clinician programmer 102 may communicate those user preferences to handheld patient programming remote 104 directly.

The example of FIG. 1 is a side view of a patient's leg 106 showing IMD 100 near the ankle and adjacent to the tibial nerve 108. IMD 100 is a leadless neurostimulation device in the example of FIG. 1. IMD 100 can be implanted through the patient's skin and cutaneous fat layer via a small incision (e.g., about one to three cm) above the tibial nerve 108 on a medial aspect of the patient's ankle. While the incision may be approximately horizontal to the length of the tibial nerve 108, other incisions or implantation techniques could be used according to physician preference. The example of FIG. 1 describes a neurostimulation implantable medical device for tibial nerve stimulation.

In the example of FIG. 1, IMD 100 may be positioned adjacent to the region defined by flexor digitorum longus and soleus in which tibial nerve 108 is contained and implanted adjacent and proximal to a fascia layer. One or more electrodes of IMD 100 may face toward tibial nerve 108. Though not shown in FIG. 1, IMD 100 may also connect to one or more leads comprising one or more electrodes (not shown in FIG. 1). In other examples, IMD 100 may include a lead which goes sub-fascia and is more directly proximal to the tibial nerve.

IMD 100 may be constructed of any polymer, metal, or composite material sufficient to house the components of IMD 100. In this example, IMD 100 may be constructed with a biocompatible housing, such as titanium or stainless steel, or a polymeric material such as silicone or polyurethane, and surgically implanted at a site in patient near the tibial nerve 108. The housing of IMD 100 may be configured to provide a hermetic seal for components, such as a rechargeable power source or a primary coin cell battery. In addition, the housing of IMD 100 may be selected of a material that facilitates receiving energy to charge the rechargeable power source.

While providing therapy, an electrical stimulation signal may be transmitted between one or more electrodes through the fascia layer. The electrical signal may be used to stimulate tibial nerve 108 which may be useful in the treatment of overactive bladder (OAB) symptoms of urinary urgency, urinary frequency and/or urge incontinence, fecal incontinence, pain, or other symptoms. The example of FIG. 1 may help relieve some symptoms of some disorders.

One type of therapy for treating bladder dysfunction includes delivery of electrical stimulation to a target tissue site within a patient to cause a therapeutic effect during delivery of the electrical stimulation. For example, delivery of electrical stimulation from IMD 100 to a target therapy site, e.g., a tissue site that delivers stimulation to modulate activity of a tibial nerve, spinal nerve (e.g., a sacral nerve), a pudendal nerve, dorsal genital nerve, an inferior rectal nerve, a perineal nerve, or branches of any of the aforementioned nerves, may provide a therapeutic effect for bladder dysfunction, such as a desired reduction in frequency of bladder contractions. In some cases, electrical stimulation of the tibial nerve may modulate afferent nerve activities to restore urinary function.

In some examples, the techniques described in this disclosure are directed to delivery of neurostimulation therapy in a non-continuous manner which may include on-cycles and off-cycles. For example, an IMD 100 may deliver neurostimulation therapy for a specified period of time followed by a specified period of time when the IMD 100 does not deliver neurostimulation (e.g., withholds delivery of neurostimulation). A period during which stimulation is delivered (an on-cycle) may include on and off periods (e.g., a duty cycle or bursts of pulses) with short inter-pulse durations of time when pulses are not delivered.

The power source of IMD 100 may include one or more capacitors, e.g., super-capacitors, batteries, or other components (e.g., chemical, or electrical energy storage devices). Example batteries may include lithium-based batteries, nickel metal-hydride batteries, or other materials. In some examples, the power source may be a primary cell battery that is replaced when depleted. In other examples, the power source may be rechargeable. The rechargeable power source may be replenished, refilled, or otherwise capable of increasing the amount of energy stored after energy has been depleted.

Handheld patient programming remote 104 may be configured to enable or disable a scheduled therapy to be delivered by IMD 100, to power IMD 100 therapy on and off, or to wake up IMD 100 from an idle state, e.g., to facilitate a wireless connection, e.g., a Bluetooth® or Bluetooth® Low Energy (BLE) connection. Handheld patient programming remote 104 may also receive information from IMD 100, such as sensed signals, temperature, errors, etc.

In one or more examples, IMD 100 may be configured to communicate with both handheld patient programming remote 104 and clinician programmer 102. In some examples, communications in accordance with the first communication protocol may be one-way communication. That is, IMD 100 may be configured to receive signals, but may not necessarily transmit signals. For example, as described in more detail, IMD 100 may receive a signal from handheld patient programming remote 104.

In examples in which handheld patient programming remote 104 is configured for powering IMD 100 therapy on and/or off, in response to the patient pressing the button, handheld patient programming remote 104 may control IMD 100 to switch between on and off. For example, if the patient presses the button when IMD 100 is on, IMD 100 powers therapy off, and if the patient presses the button when IMD 100 is off, IMD 100 powers therapy on.

In examples in which handheld patient programming remote 104 is configured for IMD wake up, in response to the patient pressing the button, handheld patient programming remote 104 may send wake up packages to IMD 100. IMD 100 may begin advertising or may increase an advertising rate from an idle rate to a higher advertising rate. IMD 100 may advertise according to a BLE protocol. For example, in response to the button press for device wake up, IMD 100 may broadcast advertisement packets at a relatively high frequency (and higher power), and a device that is going to establish a communication link with IMD 100, e.g., clinician programmer 102 or the user device, may use the advertisement packets for establishing the communication link. The advertisement packets may indicate to other devices that IMD 100 is available for establishing a communication link.

The period of time within which IMD 100 may establish the communication link or refuse establishing a communication link may be set to some programmed level or may be set to be indefinite. In some examples, the period of time within which IMD 100 may establish the communication link or refuse establishing a communication link may be set to be a reasonable time it would take to establish a communication link, e.g., 2 minutes to 5 minutes. In some examples, the patient can end an advertising session by pressing the button of handheld patient programming remote 104. In some examples, the IMD 100 may not advertise at all until the remote 104 wakes it up and it begins advertising for a short time period, thereby increasing the longevity or recharge interval of the IMD 100. In some examples, IMD 100 advertises continuously, e.g., on a periodic schedule, at a relatively low rate and increases the advertising rate to a higher rate in response to receiving the wake up packages from handheld patient programming remote 104.

In some examples, the potential function of the single actuation mechanism may be limited based on the type of IMD, e.g., the identity of the IMD. As an example, handheld patient programming remote 104 may be capable of communicating with any of a plurality of types of IMDs. In response to receiving an indication of the type of IMD, e.g., in response to receiving an indication that the IMD is a tibial stimulation device such as IMD 100, The potential functions of the single actuation device may be limited to powering the device therapy on and/or off or enabling and/or disabling a scheduled therapy and may not include device wake up.

In some examples, handheld patient programming remote 104 additionally or alternatively receives an indication of a user preference. For example, the clinician, using clinician programmer 102, may provide an indication of a preference between configuring handheld patient programming remote 104 to have a therapy power on and/or off function or a scheduled therapy enablement or disablement function. In other examples, the remote may enable or disable closed loop therapy of the IMD 100 based on sensed physiological signals or other sensors such as accelerometers, gyroscopes, temperature sensors, or combinations of sensors of IMD 100. In other examples, handheld patient programming remote 104 may switch IMD 100 from a first programming setting “Program 1” to a second programming setting “Program 2” and to a third programming setting “Program 3” before ultimately returning IMD 100 back to “Program 1,” e.g., via actuation of the actuation mechanism of IMD 100. In another example, e.g., in an example in which the IMD 100 is a targeted drug delivery (TDD) device, the remote 104 may trigger IMD 100 to deliver a bolus of drug to the patient's body or successive button presses may deliver small doses of drug up to a system defined maximum drug amount.

Based on the indication of the type of IMD and the user preference, processing circuitry of the system of FIG. 1 may configure handheld patient programming remote 104 to have a single function corresponding to the indications. For example, in response to the indication that the IMD is tibial stimulation IMD 100 and the indication of the user preference of a power on and/or off function, the handheld patient programming remote 104 may be configured to power IMD 100 therapy on and/or off.

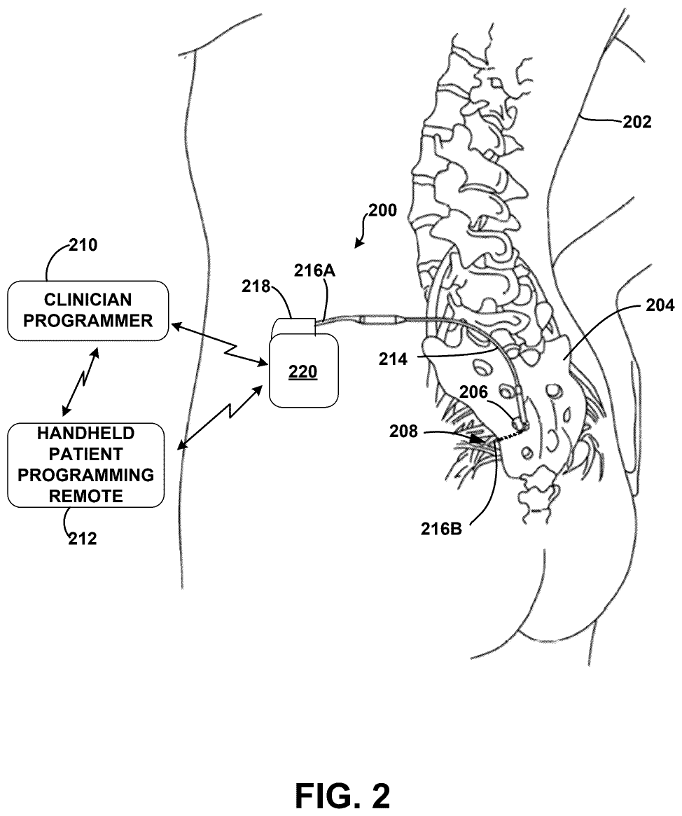

FIG. 2 is a conceptual diagram illustrating an example system including an SCS device, e.g., a sacral neuromodulation (SNM) device, a handheld patient programming remote 212, and a clinician programmer 210 according to one or more techniques of this disclosure. In the illustrated embodiment, SNM device 220 is an electrical stimulator. Accordingly, although therapy system 200 and SNM device 220 are referenced throughout the remainder of the disclosure for purposes of illustration, therapy system 200 and SNM device 220, in accordance with the disclosure, may be adapted for use in a variety of applications.

Clinician programmer 210 may be similar to clinician programmer 102 of FIG. 1 in some respects, and handheld patient programming remote 212 may be similar to or the same as handheld patient programming remote 104 of FIG. 1. Clinician programmer 210 may interact with handheld patient programming remote 212 in a similar manner to the manner described with respect to clinician programmer 102 and handheld patient programming remote 104. Handheld patient programming remote 212 may interact with SNM device 220 in a similar or the same manner as described with respect to handheld patient programming remote 104 and IMD 100. For example, handheld patient programming remote 212 may be configured to power SNM device 220 therapy on and/or off, enable and/or disable a scheduled therapy of SNM device 220, wake up SNM device 220, switch closed loop therapy on and/or off, switch between two or more therapy programs, or log patient events, e.g., urination and/or defecation.

Clinician programmer 210 and handheld patient programming remote 212 may be configured to control the operation of SNM device 220. In some examples, the clinician may use clinician programmer 210 to program SNM device 220 periodically, e.g., during clinic visits or remotely in response to a patient request or other stimulus. In some examples, clinician programmer 102 may communicate with SNM device 220 to download recorded data, to adjust one or more therapy settings, etc. Clinician programmer 210 may include a housing to enclose operational components such as a processor, memory, user interface, telemetry circuitry, and power source. Examples of clinician programmer 210 include a tablet computer, laptop, a smartphone, a dedicated handheld device, or other similar computing devices. In some examples, a patient may use handheld patient programming remote 212 to power SNM device 220 therapy on and off or to enable or disable a scheduled therapy. Handheld patient programming remote 212 may comprise a dedicated handheld device with a configurable single actuation mechanism. In some examples, a user device, such as a patient smartphone with an application (not depicted), may additionally be configured to control the operation of SNM device 220.

In some examples, clinician programmer 210 may have relatively more functionality over the operation of SNM device 220 than handheld patient programming remote 212 or the user device. For example, the clinician may set one or more stimulation amplitude limits and/or set a therapy schedule using clinician programmer 210, and the patient may be able to adjust therapy within the limits and/or disable a scheduled therapy via the user device. Additionally, the clinician, using clinician programmer 102, may select a function to be performed when the patient actuates the single actuation mechanism, e.g., a button, of handheld patient programming remote 104.

SNM device 220 is coupled to stimulation lead 214 and provides a programmable stimulation signal (e.g., in the form of electrical pulses or substantially continuous-time signals) to target stimulation site 208 via stimulation lead 214. More particularly, the programmable stimulation signal is delivered to target stimulation site 208 via one or more stimulation electrodes carried by lead 214. In some embodiments, lead 214 may also carry one or more sense electrodes to permit SNM device 220 to sense electrical signals from target stimulation site 208. Stimulation delivery and sensing may occur via the same electrodes, in some embodiments. Proximal end 216A of lead 214 may be both electrically and mechanically coupled to connector 218 of SNM device 220 either directly or indirectly (e.g., via a lead extension). In particular, conductors disposed in the lead body of lead 214 may electrically connect stimulation electrodes (and sense electrodes, if present) adjacent to distal end 216B of lead 214 to SNM device 220.

SNM device 220 may be subcutaneously implanted in the body of a patient 202 (e.g., in a chest cavity, lower back, lower abdomen, or buttocks of patient 202). In the example of FIG. 2, SNM device 220 is a neurostimulator that is implanted in patient 202 proximate to target stimulation site 208. SNM device 220 may also be referred to as a signal generator, and in the embodiment shown in FIG. 2, SNM device 220 may also be referred to as a neurostimulator. The configuration of SNM device 220 and lead 214 shown in FIG. 2 is merely exemplary. For example, in some embodiments, SNM device 220 may be coupled to two or more leads, e.g., for bilateral or multi-lateral stimulation.

In the embodiment of therapy system 200 shown in FIG. 2, target stimulation site 208 is proximate to the S3 sacral nerve, and lead 214 has been introduced into the S3 sacral foramen 206 of sacrum 204 to access the S3 sacral nerve. Stimulation of the S3 sacral nerve may help treat pelvic floor disorders, urinary control disorders, fecal control disorders, interstitial cystitis, sexual dysfunction, and pelvic pain.

Therapy system 200 may additionally or alternatively be used to provide stimulation therapy to other nerves or tissue sites of a patient. In other embodiments, target stimulation site 208 may be a location proximate to any of the other sacral nerves in patient 202 or any other suitable nerve, organ, muscle, muscle group or another suitable tissue site in patient 202, which may be selected based on, for example, the symptoms or medical condition of a particular patient. For example, therapy system 200 may be used to deliver neurostimulation therapy to a pudendal nerve, a perincal nerve or other areas of the nervous system, in which cases, lead 214 would be implanted and substantially fixed proximate to the respective nerve. As further examples, lead 214 may be positioned for temporary or chronic SCS for the treatment of pain, for peripheral neuropathy or post-operative pain mitigation, ilioinguinal nerve stimulation, intercostal nerve stimulation, gastric stimulation for the treatment of gastric mobility disorders and obesity, muscle stimulation (e.g., functional electrical stimulation (FES) of muscles), for mitigation of other peripheral and localized pain (e.g., leg pain or back pain), or for deep brain stimulation to treat movement disorders and other neurological disorders.

Handheld patient programming remote 212 may be configured to enable or disable a scheduled therapy to be delivered by SNM device 220, to power SNM device 220 therapy on and off, or to wake up SNM device 220 from an idle, e.g., low power, state, e.g., to facilitate a BLE connection. Handheld patient programming remote 212 and/or clinician programmer 210 may also receive information from SNM device 220, such as sensed signals, temperature, errors, etc.

In one or more examples, SNM device 220 may be configured to communicate with both handheld patient programming remote 212 and clinician programmer 210. In some examples, communications in accordance with the first communication protocol may be one-way communication. That is, SNM device 220 may be configured to receive signals, but may not necessarily transmit signals.

In examples in which handheld patient programming remote 212 is configured for powering SNM device 220 therapy on and/or off, in response to the patient pressing the button, handheld patient programming remote 104 may control SNM device 220 to switch between on and off. For example, if the patient presses the button when SNM device 220 is on, SNM device 220 powers therapy off, and if the patient presses the button when SNM device 220 is off, SNM device 220 powers therapy on.

In examples in which handheld patient programming remote 212 is configured for IMD wake up, in response to the patient pressing the button, handheld patient programming remote 212 may send wake up packages to SNM device 220. SNM device 220 may begin advertising or may increase an advertising rate from an idle rate to a higher advertising rate. SNM device 220 may advertise according to a BLE protocol. For example, in response to the button press for device wake up, SNM device 220 may broadcast advertisement packets at a relatively high frequency, and a device that is going to establish a communication link with SNM device 220 may use the advertisement packets for establishing the communication link. The advertisement packets may indicate to other devices that SNM device 220 is available for establishing a communication link. In some examples, handheld patient programming remote may stop sending wake up packages to SNM device 220 in response to detecting that SNM device 220 has begun sending advertisement packets or that SNM device 220 has increased a rate of sending advertisement packets from the idle rate.

The period of time within which SNM device 220 may establish the communication link or refuse establishing a communication link may be set to some programmed level or may be set to be indefinite. In some examples, the period of time within which SNM device 220 may establish the communication link or refuse establishing a communication link may be set to be a reasonable time it would take to establish a communication link. In some examples, the patient can end an advertising session by pressing the button of handheld patient programming remote 212.

In some examples, the potential function of the single actuation mechanism may be limited based on the type of IMD, e.g., the identity of the IMD. As an example, handheld patient programming remote may be capable of communicating with any of a plurality of types of IMDs. In response to receiving an indication of the type of IMD, e.g., in response to receiving an indication that the IMD is a SNM device such as SNM device 220 (as opposed to a tibial stimulation device such as IMD 100), the potential functions of the single actuation device may be limited to device wakeup or enabling and/or disabling a scheduled therapy and may not include powering the device therapy on and/or off.

In some examples, handheld patient programming remote 212 additionally or alternatively receives an indication of a user preference. For example, the clinician, using clinician programmer 210, may provide an indication of a preference between configuring handheld patient programming remote 212 to have a device wake up function or a scheduled therapy enablement or disablement function. In some examples, configuring handheld patient programming remote 212 includes configuring the function to be performed by actuating the single actuation mechanism of handheld patient programming remote 212.

Based on the indication of the type of IMD and the user preference, processing circuitry of the system of FIG. 1 may configure handheld patient programming remote 104 to have a single function corresponding to the indications. For example, in response to the indication that the IMD is an SNM device, e.g., SNM device 220, and an indication of the user preference of a device wake up function, the handheld patient programming remote 104 may be configured to wake up SNM device 220.

While the example systems provided in FIGS. 1 and 2 are directed to internal neurostimulation systems (INS), the techniques of this disclosure are not so limited. The INS systems of FIGS. 1 and 2 merely serve as examples of types of medical devices that a handheld patient programming remote, e.g., handheld patient programming remote 104 or handheld patient programming remote 212 may communicate with. In other examples, the techniques of this disclosure may apply to other medical devices without limitation. In other words, the techniques of this disclosure may apply to any medical device system in which a patient may control or adjust therapy using a programming remote.

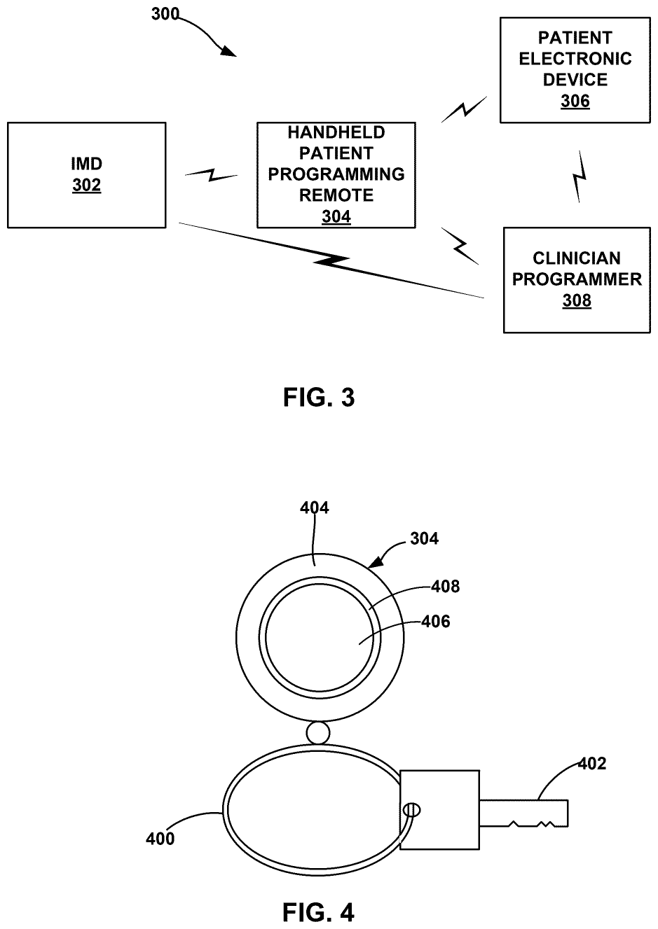

FIG. 3 illustrates system 300 in which handheld patient programming remote 304 may be used to communicate with IMD 302, e.g., IMD 100 of FIG. 1 or SNM device 220 of FIG. 2. System 300 may be similar to the system of FIG. 1 or system 200 of FIG. 2. As will be described in further detail below, handheld patient programming remote 304 is configured to communicate directly with IMD 302 and may directly program or otherwise modify therapy parameters of IMD 302 and/or provide a telemetry bridge for another programming device, such as patient electronic device 306 or clinician programmer 308. Clinician programmer 308 may be a dedicated hardware device with dedicated software for programming of IMD 302 or may be a tablet, laptop, smartphone, or other computing device running an application that enables clinician programmer 308 to program IMD 302. In another embodiment, handheld patient programming remote 304 may also be configured to provide a telemetry bridge for another programming device, such as a patient therapy manager (PTM), in addition to clinician programmer 308.

To help better meet the needs of patients, handheld patient programming remote 304 may be a relatively small device used to program the therapy delivered by IMD 302 via direct and/or indirect manipulation of handheld patient programming remote 304. Handheld patient programming remote 304 is in a separate housing from IMD 302, patient electronic device 306, and clinician programmer 308. Thus, handheld patient programming remote 304 is a self-contained telemetry device that is separate from IMD 302, patient electronic device 306, and clinician programmer 308.

Handheld patient programming remote 304 provides many roles within system 300. In each aspect, however, handheld patient programming remote 304 is configured to communicate directly with IMD 302. Handheld patient programming remote 304 is configured to bridge IMD 302 with a programming application on a separate device, whether the device is patient electronic device 306, clinician programmer 308, or another computing device. In this way, handheld patient programming remote 304 may provide an intermediary telemetry interface for IMD 302 and another device.

In some examples, handheld patient programming remote 304 is configured to transmit and receive information according to radio frequency (RF) communication protocols, such as RF communication according to the 802.11 infrared (Ir) communication and/or WiFi specification, 802.15.1 Bluetooth specification sets, or other standard or proprietary telemetry protocols.

In some embodiments, handheld patient programming remote 304 is configured to have a single function associated with a single actuation mechanism, but is configured to interface with a separate “feature-rich” programming device with substantially more programming features than handheld patient programming remote 304. Such a feature-rich device may be, for example, patient electronic device 306, clinician programmer 308, a PTM (patient therapy manager), and/or a custom hardware device. For example, handheld patient programming remote 304 may interface with patient electronic device 306 running an application to the patient to control more advanced functions, such as adjusting an amplitude of stimulation. In other embodiments, the clinician and the patient may discuss the specific needs of the patient, and the patient may elect to have more than one functionality assigned to different types of button presses such as a short press, a double short press, and/or a long press. For patients of lower technical skill levels or cognitive abilities, the clinician may only allow the patient to have a single functionality and the system may store this limitation in memory of the patient remote 304 or the IMD. The clinician may also limit an actuation mechanism of handheld patient programming remote 304 to a single function for other reasons. In other embodiments, patient electronic device 306 may configure the behavior of the different types of button presses in the programming remote 304.

As previously discussed, handheld patient programming remote 304 may include functionality that enables handheld patient programming remote 304 to directly program IMD 302. That is, in some embodiments, handheld patient programming remote 304 includes a user interface including the actuation mechanism, e.g., a button, that enables a user to power IMD 302 therapy on and/or off, to enable or disable a scheduled therapy of IMD 302, or to wake up IMD 302. In response to the button being pressed, handheld patient programming remote 304 may deliver the desired function to IMD 302.

In some examples, the button may enable the user to control IMD 302 to perform more than one function, e.g., 2 functions, 3 functions, 4 functions, or more functions. In some examples, the duration of a button press and/or the number of button presses may determine which function IMD 302 performs. As an example, pressing the button a single time may control IMD 302 to perform a first function, and pressing the button two times may control IMD 302 to perform a second function. In some examples, in addition to powering therapy on and/or off, enabling or disabling a scheduled therapy, and/or waking up IMD 302, the button may enable the user to adjust a stimulation amplitude, to switch between a closed loop therapy configuration to an open loop therapy configuration, to enable or disable stimulation during sleeping hours, to switch the time of a scheduled therapy from a first option, e.g., 8:00 a.m., to a second option, e.g., 10:00 a.m., to switch the number of days per week scheduled therapy is to be delivered from a first option, e.g., 3 days per week, to a second option, e.g., 5 days per week, to switch a duration of a scheduled therapy between a first option to a second option to a third option and back to the first option, to switch between two or more stimulation electrode combinations, to switch between a low closed loop stimulation sensitivity, a medium closed loop stimulation sensitivity, and a high closed loop stimulation sensitivity, to switch between two or more states of IMD 302, to perform an emergency reset of IMD 302, to switch to a magnetic resonance imaging (MRI) mode, to perform incremental drug delivery, or to log an event, e.g., urination, defecation, and/or meal intake.

The user interface of handheld patient programming remote 304 may provide fewer features (e.g., buttons or displays) than patient electronic device 306, clinician programmer 308, or other existing programming remotes. For example, handheld patient programming remote 304 may include an “on/off” button to turn stimulation therapy on or off (which, as discussed in further detail below, can comprise switching from “on,” e.g., a relatively higher power, to a safe mode, e.g., a relatively lower power, rather than turning therapy 100% off). In other examples, the button may be configured to enable and/or disable therapy or to wake up IMD 302. In some examples, handheld patient programming remote 304 may include two actuation mechanisms, e.g., two buttons, such as an “on/off” button and a “wake up” button. In other examples, one button may be both on & increment stim, and another button may be decrement stim & off.

Patient electronic device 306 and/or clinician programmer 308 may be configured to program handheld patient programming remote 304 during a programming phase. For example, patient electronic device 306 and/or clinician programmer 308 may include an application for configuring handheld patient programming remote 304. The application may include a field-ready configuration interface for configuring handheld patient programming remote 304 via, for example, BLE transmissions. In some examples, the BLE transmission configuration packets are encrypted, such as according to AES-256 standards. In other examples, cryptographic protections may be implemented using emerging standards such as FIPS-203 (symmetric cryptography for constrained environments), FIPS-204 (hash functions optimized for lightweight applications), or FIPS-205 (message authentication codes designed for efficiency), each intended to support secure credential and key exchanges in resource-constrained or post-quantum contexts. In some examples, handheld patient programming remote 304 may be configured to decrypt the BLE transmission configuration packets using a secure static key or a medium access control (MAC) alternative. In some examples, patient electronic device 306 and/or clinician programmer 308 may include embedded firmware, such as STM32 firmware, with modular logic to apply configuration modes and update LED state feedback. The firmware may support AES-256 electronic code book (ECB) decryption. The application may include a user interface configured to receive user input indicative of user preferences, such as a user preference for a function of the single actuation mechanism.

In some examples, patient electronic device 306 and/or clinician programmer 308 may be configured for one-way communication with handheld patient programming remote 304. For example, patient electronic device 306 and/or clinician programmer 308 may be configured to transmit packets to handheld patient programming remote 304. Handheld patient programming remote 304 may not be configured to transmit packets to patient electronic device 306 and/or clinician programmer 308. In some examples, the one-way communication may increase security of data associated with IMD 302. In some examples, patient electronic device 306 and/or clinician programmer 308 may be configured for two-way communication with handheld patient programming remote 304. Two-way communication may facilitate patient, clinician, and/or system 300 review of data of IMD 302.

In some examples, IMD 302 includes one or more LEDs to provide an indication of a state of system 300 or a device thereof. The one or more LEDs may change color or otherwise provide indications of changes to system 300. For example, when IMD 302 switches from “on” to “off,” the one or more LEDs may switch from green to red. In some examples, more and/or different LEDs may be illuminated when the IMD 302 switches from one state to another state.

Handheld patient programing remote 304 may interact with IMD 302 using telemetry protocols known in the art, such as a RF telemetry protocol, e.g., a BLE protocol, a TEL-M protocol, a Zigbee protocol, or an inductive protocol, such as a near-field communication (NFC) protocol, a Tel-N protocol, or a Tel-A protocol. Some telemetry protocols may be optimized if handheld patient programming remote 304 is placed within a certain distance of IMD 302. For example, when using a RF telemetry protocol to communicate with IMD 302, handheld patient programming remote 304 may be placed within about 2 cm to about 125 cm, such as about 13 cm, of IMD 302 during programming activities. Telemetry device 62 may be easily placed at an appropriate location during programming of IMD 302 because of its relatively small size. As mentioned above, in some examples, the actuation mechanism comprises a button. In some examples, the actuation mechanism of handheld patient programming remote 304 comprises a distance between handheld patient programming remote 304 and IMD 302. In some examples, actuating the actuation mechanism comprises placing programming remote 304 near IMD 302.

FIG. 4 is a conceptual diagram illustrating an example embodiment of handheld patient programming remote 304 with a configurable single actuation mechanism, e.g., button 406, in which handheld patient programming remote 304 is a key fob. Handheld patient programming remote 304 may be looped onto a key ring 400 that is additionally configured to receive one or more keys 402. In other embodiments, handheld patient programming remote 304 may be attached to clothing of the patient, attached to a strap secured to the patient (e.g., handheld patient programming remote 304 may be a pendent on a necklace), attached to patient electronic device 306, or placed in a pocket of the patient during programming activities. In the embodiment shown in FIG. 4, handheld patient programming remote 304 has limited functionality for programming IMD 302. In particular, handheld patient programming remote 304 includes on/off button 406 and LED 408. Button 406 is an example of a single actuation mechanism; however, other single actuation mechanisms are also possible such as sliders and sensors configured to sense signals indicative of a placement of handheld patient programming remote 304 relative to an IMD, e.g., IMD 302. Button 406 and LED 408 are electrically coupled to circuitry within housing 404 of handheld patient programming remote 304, which also defines openings for button 406 and LED 408 to extend to an outer surface. Housing 404 may be formed of any suitable material, such as a relatively hard plastic or polymer. Housing 404 may be formed of polycarbonate (PC) and/or acrylonitrile butadiene styrene (ABS). In some examples, button 406 may include a silicone over mold for tactile button interfaces and/or LEDs. In some examples, the silicone over mold is clear to facilitate LED visibility. In some examples, housing 404 may be formed via a two-step process, e.g., a two-step injection molding process. Housing 404 may be waterproof and durable. For example, housing 404 may meet IPX8 waterproofing standards. In some examples, a printed circuit board (PCB) within housing 404 may be coated in conformal parylene coating for additional waterproofing. In some examples, housing 404 may include a dedicated channel for an inductive coil to facilitate wireless communication with, e.g., IMD 302.

In the example of FIG. 4, handheld patient programming remote 304 is approximately circular in shape and may have a diameter of about, e.g., within a 10% tolerance of, 5 cm and a thickness of about 1 cm. However, the techniques of this disclosure are not so limited. Various shapes and dimensions are also possible. For example, handheld patient programming remote 304 may be approximately rectangular, e.g., 5.1 cm by 4.1 cm, and have a profile thickness of 1 cm or less. In some examples, handheld patient programming remote may include one or more rounded edges and/or corners. In general, handheld patient programming remote 304 is sized to fit comfortably in a hand of the patient.

Button 406 may be a push button, a soft-key, voice activated commands, activated by physical interactions, e.g., placing handheld patient programming remote 304 within a threshold distance from IMD 302, magnetically triggered, activated upon password authentication, a contact defined by a touch screen, or any other suitable user interface. In some embodiments, button 406 of handheld patient programming remote 304 may be reprogrammable. That is, button 406 may be reprogrammed to provide a different programming function if the needs of the patient change or if IMD 302 changes. Button 406 may be reprogrammed, for example, by clinician programmer 308 (FIG. 3) or another computing device. In some examples, handheld patient programming remote 304 may include an additional button (not depicted). In some examples, the additional button may have a different shape and/or size than button 406. In such examples, button 406 may have a first single function, e.g., IMD wakeup, and the additional button may have a second single function, e.g., IMD power on/off, therapy on/off, therapy increment/decrement, or therapy wakeup.

Button 406 may be designed to help reduce accidental activation of a programming function. For example, button 406 may be recessed from an outermost surface of housing 404. Alternatively, or additionally, the patient may be required to hold a button for a predetermined amount of time in order to activate the button, and/or there may be a hold function that prevents the buttons from being activated unless the hold function is deactivated. For example, the hold function may be activated and deactivated via manipulation of a slider bar (not shown).

Pressing button 406 when IMD 302 is on may not turn the therapy delivered by IMD 302 completely off and, instead, may change the therapy delivered by IMD 302 to a safe mode setting. The safe mode setting may or may not be equivalent to turning the therapy delivered by IMD 302 off. For some therapies and patients, turning off the therapy delivered by IMD 302 may not be safe or comfortable. A safe mode setting that defines a set of parameters that is known to provide a safe and comfortable therapy to the patient from IMD 302 may provide a better alternative than completely turning the therapy delivered by IMD 302 off. The safe mode setting may define a minimal amount of therapy that provides comfortable and safe therapy to the patient.

In the example of an implanted neurostimulator, the safe mode for the patient may be a specific combination of therapy parameters that yield a safe and comfortable therapy setting. In some embodiments, the safe mode is a preconfigured setting or a rollback to a last or last-known safe and comfortable therapy state. In one embodiment, the safe mode for an implanted neurostimulator may be to set the stimulation amplitude to zero volts. This would effectively turn off the stimulation and remove any undesirable side effects of the therapy. In some cases, eliminating the stimulation may provide discomfort to the patient if, for example, IMD 302 is used to treat pain. In such a case, the safe mode may set forth a therapy program including a relatively low current or voltage amplitude in order to provide a minimal degree of pain relief to the patient.

In some embodiments, the safe mode may be defined by allowing the patient, the clinician, a caregiver, or another qualified individual to save, via a user interface of the application running on patient electronic device 306 or via clinician programmer 308, one or more safe therapy configurations that provide the patient with safe and comfortable therapy. In some embodiments, the user interface provided on handheld patient programming remote 304 may only include means for programming IMD 302 to enter a defined safe mode, e.g., button 406 may be an “off” button and may not be capable of turning IMD 302 “on.” The safe mode settings may be saved within handheld patient programming remote 304, which may include any volatile or non-volatile media, such as a RAM, ROM, NVRAM, EEPROM, flash memory, and the like to store the safe mode settings for IMD 302. Alternatively, the settings for the safe mode of IMD 302 may be stored in IMD 302, and handheld patient programming remote 304 may provide instructions to IMD 302 to access and implement the stored safe mode setting, rather than sending the actual safe mode settings themselves.

LED 408 (or another LED) may provide confirmation to a user that an operation has been carried out or that an input via button 406 or another user interface, e.g., the user interface of patient electronic device 306, has been received. For example, when button 406 is depressed by the patient, and a programming signal is sent to IMD 302 to turn therapy on or off, LED 408 may be activated in order to provide positive feedback to the patient regarding the successfully sent programming signal. Handheld patient programming remote 408 may include more than one LED 408. In the example of FIG. 4, LED 408 is disposed around button 406. In some examples, LED 408 may be disposed on button 406. LED 408 may be configured such that LED 408 provides visual cues for device status, therapy parameters, etc. LED 408 may be configured to be visible in low-light conditions.

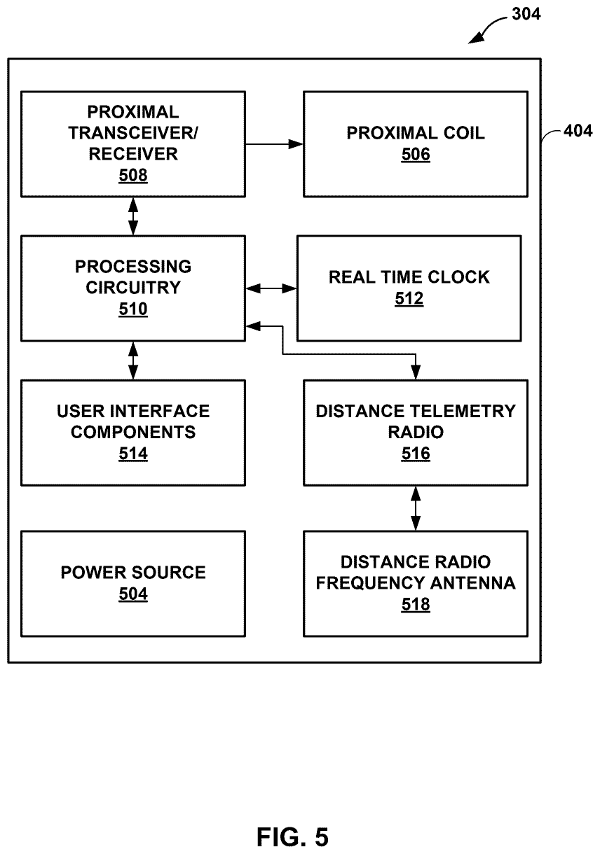

FIG. 5 is a block diagram illustrating an example configuration of handheld patient programming remote 304 according to one or more techniques of this disclosure. Handheld patient programming remote 304 includes components disposed within housing 404 (also shown in FIG. 4). Handheld patient programming remote 304 may include a proximal transceiver 508 and proximal coil 506 for communicating with IMD 302, patient electronic device 306, clinician programmer 308, and any other device. In some examples, e.g., in examples in which IMD 302 is configured for medium to long range communication, e.g., BLE, Bluetooth®, medical implant communication system (MICS), or proprietary communication, handheld patient programming remote 304 additionally includes distance telemetry radio 516 and distance frequency antenna 518. Handheld patient programming remote 304 also includes processing circuitry 510, user interface components 514, real time clock 512, and power source 504. In some embodiments, handheld patient programming remote 304 additionally includes a memory (not shown).

As described previously, proximal transceiver 508 may, via proximal coil 506, communicate with IMD 302, patient electronic device 306, and/or clinician programmer 308 using telemetry protocols known in the art, such as RF telemetry techniques. Distance radio frequency antenna 518 may be used to receive signals from IMD 302, patient electronic device 306, and/or clinician programmer 308. Alternatively, a proximal coil 506 may be used to receive signals from IMD 302.

Processing circuitry 510 may include a microprocessor, a controller, a DSP, an ASIC, an FPGA, discrete logic circuitry, or the like. Processing circuitry 510 controls communication between handheld patient programming remote 304 and IMD 302 and may also control communication between handheld patient programming remote 304 and patient electronic device 306 and/or clinician programmer 308. Processing circuitry 510 may also be used to “translate” signals received via telemetry protocol used by IMD 302 to a signal to the telemetry protocol used by patient electronic device 306 and/or clinician programmer 308, or vice versa. Processing circuitry 510 is also configured to execute software that may be stored within a memory of handheld patient programming remote 304. The software may include, for example, IMD 302 programming applications. Additionally, processing circuitry 510 may transfer information to and from the memory.

In examples in which handheld patient programming remote 304 includes a memory, the memory may include any volatile or non-volatile media, such as a RAM, ROM, NVRAM, EEPROM, flash memory, and the like. The memory may store data received from IMD 302. For example, the memory may store data relating to the status and/or programming history of IMD 302 or physiological parameter values determined by sensors coupled to IMD 302. Data may be stored in the memory until patient electronic device 306 or clinician programmer 308 requests to receive the data from handheld patient programming remote 304. In addition, processing circuitry 510 may extract information from the data received from IMD 302, such as to provide an alert function for IMD 302.

Handheld patient programming remote 304 includes a real time clock 512, which may facilitate synchronization of handheld patient programming remote 304, IMD 302, patient electronic device 306, and clinician programmer 308. Additionally, in examples in which handheld patient programming remote includes a memory, real time clock 512 may facilitate timestamping of data. Real time clock 512 may also be used to inform time-based protocols, such as limiting access to certain functions during specific hours of the day.

Handheld patient programming remote 404 includes user interface components 514, which may include, as examples, button 406 and LED 408, as depicted in FIG. 4. Processing circuitry 510 may process one or more inputs from button 406, and may change one or more LED displays based on the input from button 406 or other information processing circuitry 510 receives. User interface components 514 may be configurable, and user interface components may include a configuration single actuation mechanism, e.g., button 406. In some examples, using a clinician programmer, e.g., clinician programmer 308, the clinician may configure a function to be performed when the patient presses button 406. The function may be a wake up function, an IMD therapy power on/off function, or a therapy enablement/disablement function.

Handheld patient programming remote 304 also includes power source 504, which may be a battery, e.g., a coin cell battery. In embodiments in which power source 504 is rechargeable, handheld patient programming remote 304 may include a recharge interface, such as a USB port, that may be connected to a power source for recharging of handheld patient programming remote 304. Due to the relatively small size and reduced complexity of handheld patient programming remote 304 relative to other existing handheld patient programming remote s, handheld patient programming remote 304 may need to be recharged relatively infrequently.

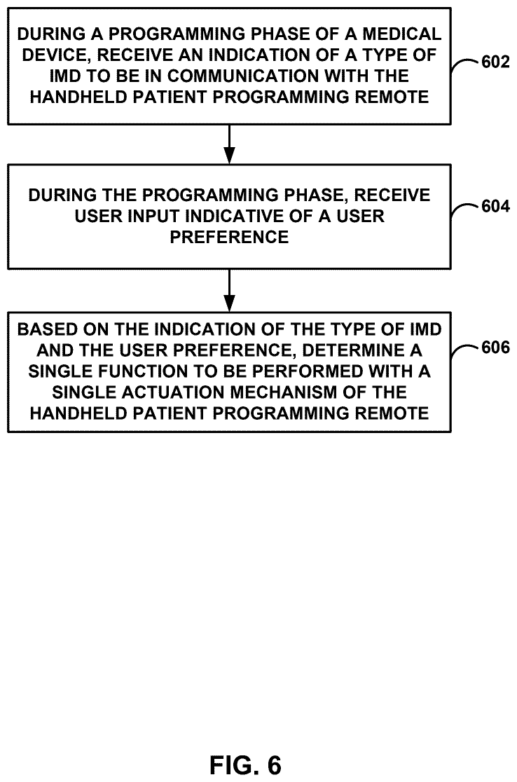

FIG. 6 is a flow chart illustrating an example operation for configuring a single actuation mechanism of a handheld patient programming remote, e.g., handheld patient programming remote 104 of FIG. 1, handheld patient programming remote 212 of FIG. 2, or handheld patient programming remote 304 of FIGS. 3-5 according to one or more techniques of this disclosure. As discussed above, handheld patient programming remote 104, handheld patient programming remote 212, and handheld patient programming remote 304 may all be the same handheld patient programming remote or substantially similar handheld patient programming remotes. Although the example operation of FIG. 6 is described with respect to handheld patient programming remote 304 of FIGS. 3-5 and the associated system 300, the techniques of the example operation may be applied to any of the example systems of this disclosure and any other systems including a medical device, e.g., an IMD or an external device, and a handheld patient programming remote.

During a programming phase, processing circuitry of system 300, e.g., processing circuitry 510 of handheld patient programming remote 304, or processing circuitry of clinician programmer 308, receives an indication of a type of medical device, e.g., a type of IMD, to be in communication with handheld patient programming remote 304 (602). In some examples, processing circuitry 510 receives the indication of the type of medical device to be in communication with handheld patient programming remote 304 from a user device, such as clinician programmer 308 or patient electronic device 306. For example, clinician programmer 308 may include an application, e.g., a SwiftUI-based iOS application, for configuring handheld patient programming remote 304. The application may include a field-ready configuration interface for configuring handheld patient programming remote 304 via, for example, BLE transmissions. In some examples, the BLE transmission configuration packets are encrypted, such as according to AES-256 standards. In some examples, handheld patient programming remote 304 may be configured to decrypt the BLE transmission configuration packets using a secure static key or a MAC alternative. In some examples, patient electronic device 306 and/or clinician programmer 308 may include embedded firmware, such as STM32 firmware, with modular logic to apply configuration modes and update LED state feedback. The firmware may support AES-256 ECB decryption. The application may include a user interface configured to receive user input indicative of user preferences, such as a user preference for a function of the single actuation mechanism. The user interface may additionally facilitate mode toggling. In some examples, the user interface includes one or more confirmation buttons allowing the user to confirm a function selection and/or medical device type selection.

In some examples, patient electronic device 306 and/or clinician programmer 308 may be configured for one-way communication with handheld patient programming remote 304. For example, patient electronic device 306 and/or clinician programmer 308 may be configured to transmit packets to handheld patient programming remote 304. Handheld patient programming remote 304 may not be configured to transmit packets to patient electronic device 306 and/or clinician programmer 308. In some examples, the one-way communication may increase security of data associated with IMD 302. In some examples, patient electronic device 306 and/or clinician programmer 308 may be configured for two-way communication with handheld patient programming remote 304. Two-way communication may facilitate patient, clinician, and/or system 300 review of data of IMD 302. The type of medical device, e.g., IMD, to be in communication with handheld patient programming remote 304 may be one of a plurality of types of medical devices, such as a tibial stimulation device, an SNM device, a pudendal stimulation device, an SCS device, a drug pump, a deep brain stimulation device, or any other type of external medical device or IMD configured to deliver therapy to a patient.

In some examples, the processing circuitry of clinician programmer 308 receives the indication of the type of medical device to be in communication with handheld patient programming remote 304 during implantation, shortly after implantation of IMD 302, e.g., during programming immediately after implantation or within a few days or weeks of implantation, or during a subsequent clinic visit when the clinician is programming IMD 302. In other examples, the processing circuitry of clinician programmer 308 receives the indication of the type of medical device to be in communication with handheld patient programming remote 304 during a manufacturing or distribution process, i.e., before implantation of IMD 302. In other examples, the type of medical device is predetermined. In other words, in some examples, handheld patient programming remote 304 is manufactured to communicate with only one type of medical device.

In some examples, a single actuation mechanism may be configured to perform any one of a plurality of functions. The plurality of functions may include, as examples, powering the medical device, e.g., IMD 302, therapy on/off, enabling/disabling a scheduled therapy of IMD 302, and waking up IMD 302. Based on the indication of the type of medical device to be in communication with handheld patient programming remote 304, the processing circuitry of clinician programmer 308 may narrow down the plurality of functions to a subset of functions. For examples, in response to determining the type of medical device is a tibial stimulation device, the processing circuitry of clinician programmer 308 may determine the subset of functions to include powering IMD 302 therapy on/off and enabling/disabling a scheduled therapy of IMD 302 and may exclude the function of waking up IMD 302, which may in some examples not be applicable to the tibial stimulation device.

During the programming phase, the processing circuitry of clinician programmer 308 receives user input indicative of a user preference (604). In some examples, the clinician provides the user input using the user interface of clinician programmer 308. In some examples, the patient provides the user input using handheld patient programming remote 304. The clinician may provide the user input based on a conversation with the patient regarding the patient's symptoms or desired outcomes of the use of IMD 302. In some examples, the user input includes a planned therapy program. In some examples, the user input includes patient symptoms. In some examples, the user input includes an explicit indication of a preferred single function to be performed with a single actuation mechanism of handheld patient programming remote 304. In examples in which IMD 302 comprises a tibial stimulation device, therapy may include a powering IMD 302 therapy on and off on an as-needed basis configuration or may include a scheduled therapy program configuration, e.g., delivering stimulation for five minutes every hour. Processing circuitry 510 may receive user input indicative the configuration of the therapy.

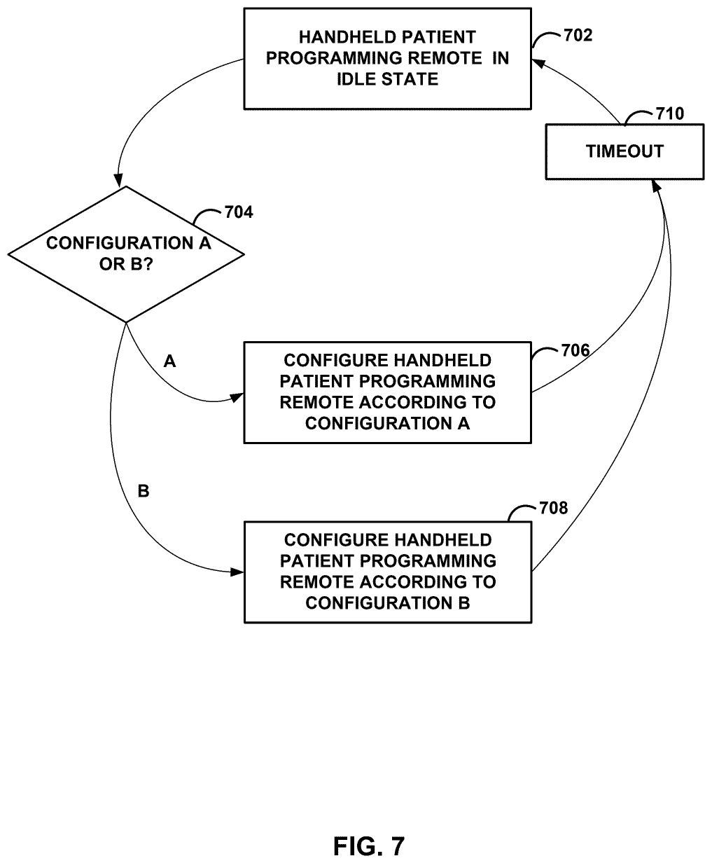

Based on the indication of the type of medical device and/or the user preference, the processing circuitry of clinician programmer 308 determines the single function to be performed with the single actuation mechanism of handheld patient programming remote 304 (606). In examples in which the processing circuitry of clinician programmer 308 determines the subset of functions based on the type of medical device, the processing circuitry of clinician programmer 308 determines a function of the subset of functions that will be performed with the single actuation mechanism of the handheld patient programming remote based on the user input indicative of the user preference. In examples in which IMD 302 is a tibial stimulation device and the user input indicates that the therapy includes powering IMD 302 therapy on and off on an as-needed basis, the processing circuitry of clinician programmer 308 may determine the single function to be the power on and off function. In examples in which the user input indicates that the therapy is configured to be delivered on a schedule, the processing circuitry of clinician programmer 308 may determine the single function to be the therapy enablement and disablement function. In examples in which handheld patient programming remote 304 includes an additional actuation mechanism, the processing circuitry of clinician programmer 308 may repeat the example operation of FIG. 7 for the additional actuation mechanisms of handheld patient programming remote 304.

FIG. 7 is a flow chart illustrating an example operation for determining a type of medical device to be in communication with a handheld patient programming remote according to one or more techniques of this disclosure. In some examples, FIG. 7 comprises a specific example of determining a single function to be performed with a single actuation mechanism of a handheld patient programming remote, as described with respect to block 606 of FIG. 6. Although the example operation of FIG. 7 is described with respect to handheld patient programming remote 304 of FIGS. 3-5 and the associated system 300, the techniques of the example operation may be applied to any of the example systems of this disclosure and any other systems including a medical device, e.g., an IMD or an external device, and a handheld patient programming remote.

Handheld patient programming remote 304 may initially be in an idle state (702). In some examples, the idle state may be associated with a relatively low advertising rate, a sleep state, and/or some other state associated with reduced battery drain. Processing circuitry of system 300, e.g., processing circuitry 510 of handheld patient programming remote 304 or the processing circuitry of clinician programmer 308, receives an indication of a configuration, and based on the indication, the processing circuitry of clinician programmer 308 determines whether to select configuration A or configuration B (704). There may be any number of configurations, such as three configurations, four configurations, five configurations, or more configurations. In some examples, the indication comprises an indication of the type of medical device to be in communication with handheld patient programming remote 304 and an indication of a user input indicative of a user preference. In examples in which the processing circuitry of clinician programmer 308 determines to select configuration A (“A” of 704), the processing circuitry of clinician programmer 308 configures handheld patient programming remote 304, e.g., operates, causes to behave, or interfaces with IMD 302, according to configuration A. In some examples, configuration A may correspond to a device wake up function. In examples in which the processing circuitry of clinician programmer 308 determines to select configuration B (“B” of 704), the processing circuitry of clinician programmer 308 configures handheld patient programming remote according to configuration B. In some examples, configuration B may correspond to a power on and off function or a therapy enablement or disablement function. In some examples, after a threshold period of time has elapsed, handheld patient programming remote 304 times out (710) and reenters an idle state (702).

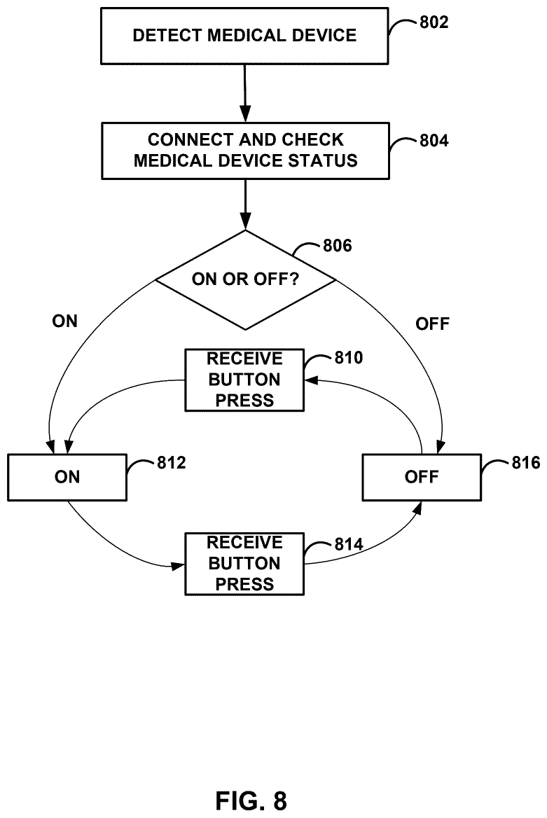

FIG. 8 is a flow chart illustrating an example operation for a handheld patient programming remote performing a power on and off function according to one or more techniques of this disclosure. Although the example operation of FIG. 8 is described with respect to handheld patient programming remote 304 of FIGS. 3-5 and the associated system 300, the techniques of the example operation may be applied to any of the example systems of this disclosure and any other systems including a medical device, e.g., an IMD or an external device, and a handheld patient programming remote.

Processing circuitry of system 300, e.g., processing circuitry 510 of handheld patient programming remote 304 or the processing circuitry of clinician programmer 308 detects IMD 302 (802). In some examples, detecting IMD 302 comprises receiving an advertising signal from IMD 302. Upon detecting IMD 302, handheld patient programming remote connects to IMD 302 and determines a medical device status (804). The medical device status may be an indication of whether IMD 302 is powered on or off (e.g., whether IMD 302 is delivering stimulation or not delivering stimulation/delivering stimulation at a “safe mode”). The medical device status may be an indication of a battery percentage of IMD 302, an indication of whether IMD 302 is operating normally, an indication of whether IMD 302 is configured to delivery scheduled therapy, an indication of when the next scheduled therapy will take place, or, in examples in which the medical device comprises a TDD device, how much of a bolus drug remains in a reservoir of the TDD. In some examples, the medical device status may indicate to the patient to check the medical device status on patient electronic device 306 for more information or for instructions.

In response to a determination that IMD 302 is on (“ON” of 806), processing circuitry 510 enters an on mode (812). Processing circuitry 510 receives an indication that the patient is actuating the actuation mechanism (e.g., the button) of handheld patient programming remote 304 (814). In response to receiving the indication, processing circuitry 510 communicates with IMD 302 to turn IMD 302 off and enters an off mode (816).

In response to a determination that IMD 302 is off (“OFF” of 806), processing circuitry 510 enters the off mode (816). Processing circuitry 510 receives an indication that the patient is actuating the actuation mechanism (e.g., the button) of handheld patient programming remote 304 (810). In response to receiving the indication, processing circuitry 510 communicates with IMD 302 to turn IMD 302 on and enters an on mode (812). Processing circuitry 510 may switch between on mode and off mode responsive to receiving indications of button presses indefinitely until the connection between IMD 302 and handheld patient programming remote 304 is lost.

In examples in which the single function of the actuation mechanism of handheld patient programming remote is enabling and disabling therapy, on mode and off mode may instead comprise enable mode and disable mode, respectively, and processing circuitry 510 may switch between enable mode and disable mode responsive to receiving indications of button presses indefinitely until the connection between IMD 302 and handheld patient programming remote 304 is lost.

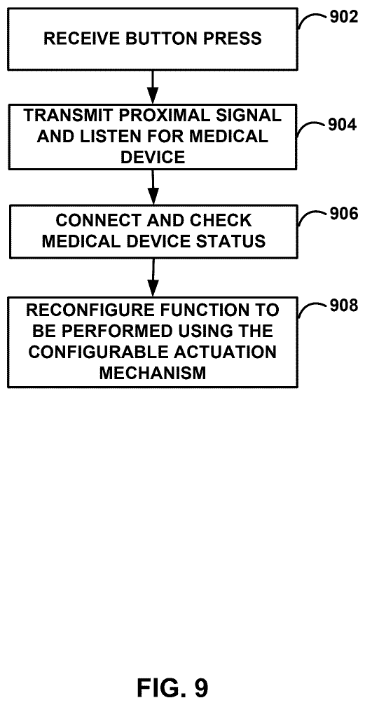

FIG. 9 is a flow chart illustrating an example operation for a handheld patient programming remote performing a wake up function according to one or more techniques of this disclosure. Although the example operation of FIG. 9 is described with respect to handheld patient programming remote 304 of FIGS. 3-5 and the associated system 300, the techniques of the example operation may be applied to any of the example systems of this disclosure and any other systems including a medical device, e.g., an IMD or an external device, and a handheld patient programming remote.

Processing circuitry 510 receives an indication that the patient has actuated the actuation mechanism, e.g., that the patient has placed handheld patient programming remote 304 within a threshold distance from IMD 302 or that the patient has pressed the button of handheld patient programming remote 304 (902). Processing circuitry 510 may transmit a proximal signal via proximal coil 506 and using proximal transceiver 508 and may listen for advertisements from IMD 302 using distance telemetry radio 516 via distance radio frequency antenna 518 (904).

In some examples, processing circuitry 510 continuously transmits the proximal signal using proximal transceiver 508 via proximal coil 506 and does not listen for advertisements from IMD 302. In such examples, the patient may actuate the actuation mechanism to cause IMD 302 to send advertisements to another device of system 300, such as patient electronic device 306. Processing circuitry 510 may stop transmitting the proximal signal after a threshold period of time has elapsed.

Upon receiving an advertisement from IMD 302, processing circuitry 510 may connect to IMD 302 and check a status of IMD 302 (906). The status of IMD 302 may comprise whether IMD 302 is on or off, whether IMD 302 is functioning as expected, or whether therapy is enabled or disabled. In some examples, processing circuitry 510 may switch the single function to be performed using the actuation mechanism, e.g., the button (908). For example, upon connecting to IMD 302, when the patient actuates the actuation mechanism, the actuation mechanism may power the medical device on/off or enable/disable scheduled therapy until the connection is lost, at which point the single function may switch back to a device wake up function.

In some examples, IMD 302, which may be substantially similar to or the same as IMD 100, may be configured to, upon connecting to handheld patient programming remote 304 and/or patient electronic device 306, maintain the BLE connection with patient electronic device 306 continuously. An application on patient electronic device 306 may be configured to communicate, e.g., periodically communicate, with handheld patient programming remote 304 to instruct handheld patient programming remote 304 to sting or communicate with IMD 302.

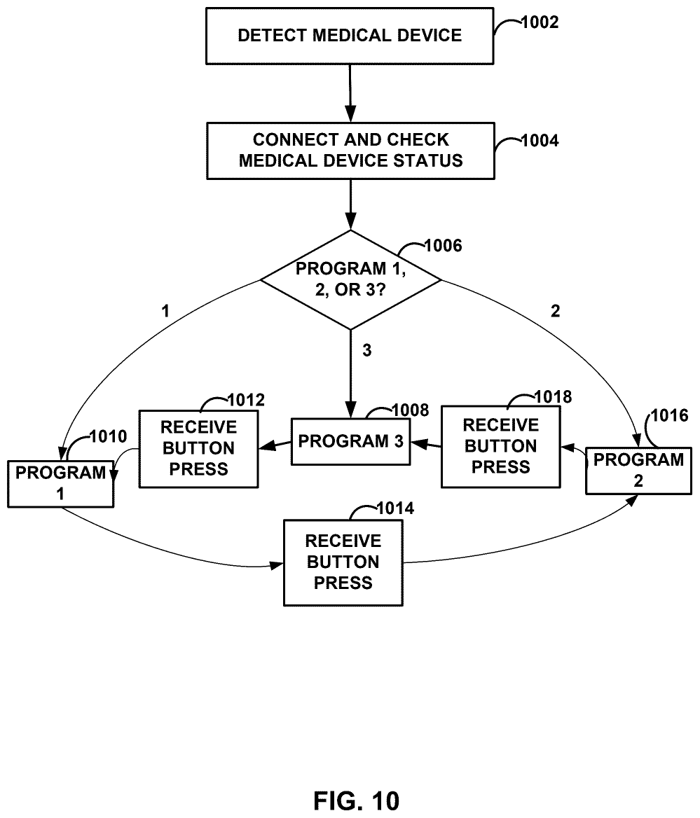

FIG. 10 is a flow chart illustrating an example operation for a handheld patient programming remote performing a switching between programs function according to one or more techniques of this disclosure.

Although the example operation of FIG. 10 is described with respect to handheld patient programming remote 304 of FIGS. 3-5 and the associated system 300, the techniques of the example operation may be applied to any of the example systems of this disclosure and any other systems including a medical device, e.g., an IMD or an external device, and a handheld patient programming remote.

Processing circuitry of system 300, e.g., processing circuitry 510 of handheld patient programming remote 304 or the processing circuitry of clinician programmer 308 detects IMD 302 (1002). In some examples, detecting IMD 302 comprises receiving an advertising signal from IMD 302. Upon detecting IMD 302, handheld patient programming remote connects to IMD 302 and determines a medical device status (1004). The medical device status may be an indication of whether IMD 302 is powered on or off (e.g., whether IMD 302 is delivering stimulation or not delivering stimulation/delivering stimulation at a “safe mode”). The medical device status may be an indication of a battery percentage of IMD 302, an indication of whether IMD 302 is operating normally, an indication of whether IMD 302 is configured to delivery scheduled therapy, an indication of when the next scheduled therapy will take place, or, in examples in which the medical device comprises a TDD device, how much of a bolus drug remains in a reservoir of the TDD. In some examples, the medical device status may indicate to the patient to check the medical device status on patient electronic device 306 for more information or for instructions.

In some examples, IMD 302 may be configured to deliver therapy via one or more programs. The patient may need or want to switch between therapy programs at various times or in various situations. As an example, during sleep, the patient may want IMD 302 to deliver therapy using a first program, e.g., program 1. The patient may want IMD 302 to deliver therapy using a second program, e.g., program 2, during while the patient is at work, and the patient may want IMD 302 to deliver therapy using a third program, e.g., program 3, while the patient is at home. The function of the actuation mechanism of handheld patient programming remote 304 may be to switch between the therapy programs.

Processing circuitry 510 may communicate with IMD 302 to cycle between therapy programs, going from program 1 to 2, 2 to 3, and 3 to 1 in response to receiving indications of the actuation mechanism being actuated. For example, in response to a determination that IMD 302 is operating using program 1, (“1” of 1006), processing circuitry 510 enters program 1 mode (1010). Processing circuitry 510 receives an indication that the patient is actuating the actuation mechanism (e.g., the button) of handheld patient programming remote 304 (1014). In response to receiving the indication, processing circuitry 510 communicates with IMD 302 to switch from program 1 to program 2 and enters program 2 mode (1016).

In response to a determination that IMD 302 is operating using program 2, (“2” of 1006), processing circuitry 510 enters program 2 mode (1016). Processing circuitry 510 receives an indication that the patient is actuating the actuation mechanism (e.g., the button) of handheld patient programming remote 304 (1018). In response to receiving the indication, processing circuitry 510 communicates with IMD 302 to switch from program 2 to program 3 and enters program 3 mode (1008).

In response to a determination that IMD 302 is operating using program 3, (“3” of 1006), processing circuitry 510 enters program 3 mode (1008). Processing circuitry 510 receives an indication that the patient is actuating the actuation mechanism (e.g., the button) of handheld patient programming remote 304 (1012). In response to receiving the indication, processing circuitry 510 communicates with IMD 302 to switch from program 3 to program 1 and enters program 1 mode (1010).

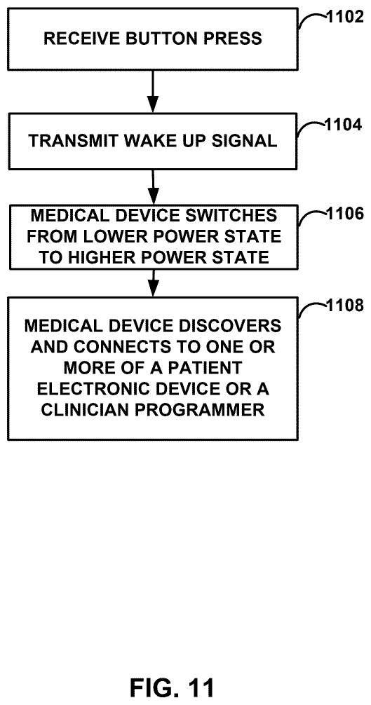

FIG. 11 is a flow chart illustrating an example operation for a handheld patient programming remote performing a wake up function according to one or more techniques of this disclosure. Although the example operation of FIG. 11 is described with respect to handheld patient programming remote 304 of FIGS. 3-5 and the associated system 300, the techniques of the example operation may be applied to any of the example systems of this disclosure and any other systems including a medical device, e.g., an IMD or an external device, and a handheld patient programming remote.

Processing circuitry 510 receives an indication that the patient has actuated the actuation mechanism, e.g., that the patient has placed handheld patient programming remote 304 within a threshold distance from IMD 302 or that the patient has pressed the button of handheld patient programming remote 304 (1102). Processing circuitry 510 may transmit a proximal signal, e.g., a wake up package, via proximal coil 506 and using proximal transceiver 508 (1104).

The patient may actuate the actuation mechanism to cause IMD 302 to send advertisements to another device of system 300, such as patient electronic device 306 and/or clinician programmer 308. Processing circuitry 510 may stop transmitting the proximal signal after a threshold period of time has elapsed.

Upon receiving the wake up packages from handheld patient programming remote 304, IMD 302 switches from a relatively low power state to a higher power state (1106). In some examples, in the relatively lower power state, IMD 302 advertises at a relatively slow rate. In other examples, in the relatively lower power state, IMD 302 does not advertise. In the higher power state, IMD 302 advertises at a faster rate. In some examples, advertising at the faster rate facilitates the establishment of a connection faster. IMD 302 discovers and connects to one or more of patient electronic device 306 or clinician programmer 308 (1108).

In some examples, IMD 302, which may be substantially similar to or the same as IMD 100, may be configured to, upon connecting to handheld patient programming remote 304 and/or patient electronic device 306, maintain the BLE connection with patient electronic device 306 continuously. An application on patient electronic device 306 may be configured to communicate, e.g., periodically communicate, with handheld patient programming remote 304 to instruct handheld patient programming remote 304 to sting or communicate with IMD 302. In some examples, the connection between IMD 302 and patient electronic device 306 and/or clinician programmer 308 may time out after a threshold period of time has elapsed.

Example 1. A medical device system comprising: a handheld patient remote, the handheld patient programming remote having a configurable single actuation mechanism; and processing circuitry configured to: during a programming phase of a medical device, receive an indication of a type of the medical device to be in communication with the handheld patient programming remote; during the programming phase, receive user input indicative of a user preference; based on the indication of the type of medical device and the user preference, determine a function to be performed with the single actuation mechanism of the handheld patient programming remote; and configure the single actuation mechanism to perform, in response to patient action, the determined operation.

Example 2. The medical device system of example 1, wherein the handheld patient programming remote is configured to perform the determined function in response to one of: a button press, wherein the handheld patient programming remote comprises a single button; or the handheld patient programming remote being within a threshold distance from the medical device.

Example 3. The medical device system of any of examples 1 and 2, wherein the handheld patient programming remote is configured to communicate with the medical device via one of: radio frequency communication; or inductive communication.

Example 4. The medical device system of any of examples 1-3, wherein the function to be performed with the single actuation mechanism of the handheld patient programming remote comprises one of: a wake up function; a scheduled therapy enablement and disablement function; or a medical device power on and off function.

Example 5. The medical device system of any of examples 1-4, wherein the single actuation mechanism comprises a button, and wherein, during a therapy phase, the handheld patient programming remote is configured to: detect the medical device; wirelessly connect to the medical device; determine a status of the medical device; determine whether the medical device is one of: on or off, wherein if the medical device is on, the handheld patient programming remote is configured to switch the medical device from on to off in response to a first button press of the button, and wherein if the medical device is off, the handheld patient programming remote is configured to switch the medical device from off to on in response to a second button press of the button; or enabled or disabled, wherein if therapy of the medical device is enabled, the handheld patient programming remote is configured to switch the therapy of the medical device from enabled to disabled in response to a first button press of the button, and wherein if the therapy of the medical device is disabled, the handheld patient programming remote is configured to switch the therapy of the medical device from disabled to enabled in response to a second button press of the button.

Example 6. The medical device system of any of examples 1-4, wherein the single actuation mechanism comprises a button, and wherein during a therapy phase, the handheld patient programming remote is configured to: receive an indication of a first button press of the button; transmit a proximal signal; and wirelessly connect to the medical device.

Example 7. The medical device system of any of examples 1-4, wherein the single actuation mechanism comprises a button, and wherein during a therapy phase, the handheld patient programming remote is configured to: receive an indication of a first button press of the button; transmit a proximal signal; and switch to an idle state in response to one of: a threshold period of time passing; or receiving an indication of a second button press.

Example 8. The medical device system of example 6, wherein, during the therapy phase, the processing circuitry is configured to: in response to wirelessly connecting to the medical device, determine to reconfigure the configurable single actuation mechanism.

Example 9. The medical device system of any of examples 1-8, further comprising: a patient electronic device, wherein the patient electronic device is configured to adjust one or more therapy configurations of the medical device.

Example 10. The medical device system of any of examples 1-9, wherein a housing of the handheld patient programming remote has a diameter within 5 centimeters and a thickness is within 1 centimeter.

Example 11. The medical device system of any of examples 1-10, wherein the handheld patient programming remote comprises one or more light emitting diodes (LEDs).

Example 12. The medical device system of any of examples 1-11, wherein the medical device comprises one of: a tibial neuromodulation device; a pudendal stimulation device; a deep brain stimulation device; a drug pump; a spinal cord stimulation device; or a sacral neuromodulation device.

Example 13. The medical device system of any of examples 1-12, wherein the processing circuitry of the system comprises processing circuitry of a clinician programming device of the system.

Example 14. A method comprising: receiving, by processing circuitry of a medical device system and during a programming phase of a medical device, an indication of a type of the medical device to be in communication with a handheld patient programming remote, the handheld patient programming remote having a configurable single actuation mechanism; receiving, by the processing circuitry and during the programming phase, user input indicative of a user preference; determining, by the processing circuitry and based on the indication of the type of medical device and the user preference, a function to be performed with the single actuation mechanism of the handheld patient programming remote; and configuring, by the processing circuitry, the single actuation mechanism to perform, in response to patient action, the determined operation.

Example 15. The method of example 14, wherein the handheld patient programming remote is configured to perform the determined function in response to one of: receiving an indication of a button press, wherein the handheld patient programming remote comprises a single button; or determining the handheld patient programming remote is within a threshold distance from the medical device.

Example 16. The method of any of examples 14 and 15, wherein the handheld patient programming remote is configured to communicate with the medical device via one of: radio frequency communication; or inductive communication.

Example 17. The method of any of examples 14-16, wherein the function to be performed with the single actuation mechanism of the handheld patient programming remote comprises one of: a wake up function; a scheduled therapy enablement and disablement function; or an medical device power on and off function.

Example 18. The method of any of examples 14-17, wherein the single actuation mechanism comprises a button, the method further comprising: detecting, by the handheld patient programming remote and during a therapy phase, the medical device; wirelessly connecting, by the handheld patient programming remote and during the therapy phase, to the medical device; determining, by the handheld patient programming remote and during the therapy phase, a status of the medical device; determining, by the handheld patient programming remote and during the therapy phase, whether the medical device is one of: on or off, wherein if the medical device is on, the handheld patient programming remote is configured to switch the medical device from on to off in response to a first button press of the button, and wherein if the medical device is off, the handheld patient programming remote is configured to switch the medical device from off to on in response to a second button press off the button; or enabled or disabled, wherein if therapy of the medical device is enabled, the handheld patient programming remote is configured to switch the therapy of the medical device from enabled to disabled in response to a first button press of the button, and wherein if the therapy of the medical device is disabled, the handheld patient programming remote is configured to switch the therapy of the medical device from disabled to enabled in response to a second button press of the button.

Example 19. The method of any of examples 14-17, wherein the single actuation mechanism comprises a button, the method further comprising: receiving, by the handheld patient programming remote and during a therapy phase, an indication of a first button press of the button; transmitting, by the handheld patient programming remote and during the therapy phase, a proximal signal; and wirelessly connecting, by the handheld patient programming remote and during the therapy phase, to the medical device.

Example 20. The method of any of examples 14-17, wherein the single actuation mechanism comprises a button, the method further comprising: receiving, by the handheld patient programming remote and during a therapy phase, an indication of a first button press of the button; transmitting, by the handheld patient programming remote and during the therapy phase, a proximal signal; and switching, by the handheld patient programming remote and during the therapy phase, to an idle state in response to one of: a threshold period of time passing; or receiving an indication of a second button press.

Example 21. The method of example 19, further comprising: determining, by the processing circuitry and in response to wirelessly connecting to the medical device, to reconfigure the configurable single actuation mechanism.

Example 22. The method of any of examples 14-21, wherein the medical device system further comprises: a patient electronic device, wherein the patient electronic device is configured to adjust one or more therapy configurations of the medical device.

Example 23. The method of any of examples 14-22, wherein a housing of the handheld patient programming remote has a diameter within 5 centimeters and a thickness is within 1 centimeter.

Example 24. The method of any of examples 14-23, wherein the handheld patient programming remote comprises one or more light emitting diodes (LEDs).

Example 25. The method of any of examples 14-24, wherein the medical device comprises one of: a tibial neuromodulation device; a pudendal stimulation device; a deep brain stimulation device, a drug pump; a spinal cord stimulation device; or a sacral neuromodulation device.

Example 26. The method of any of examples 14-25, wherein the processing circuitry of the system comprises processing circuitry of a clinician programming device of the system.

Example 27. A non-transitory computer-readable medium storing instructions that when executed cause processing circuitry to: during a programming phase of a medical device, receive an indication of a type of the medical device to be in communication with a handheld patient programming remote, the handheld patient programming remote having a configurable single actuation mechanism; during the programming phase, receive user input indicative of a user preference; based on the indication of the type of medical device and the user preference, determine a function to be performed with the single actuation mechanism of the handheld patient programming remote; and configure the single actuation mechanism to perform, in response to patient action, the determined operation.

The techniques described in this disclosure may be implemented, at least in part, in hardware, software, firmware or any combination thereof. For example, various aspects of the described techniques may be implemented within one or more processors or processing circuitry, including one or more microprocessors, digital signal processors (DSPs), application specific integrated circuits (ASICs), field programmable gate arrays (FPGAs), or any other equivalent integrated or discrete logic circuitry, as well as any combinations of such components. The term “processor” or “processing circuitry” may generally refer to any of the foregoing logic circuitry, alone or in combination with other logic circuitry, or any other equivalent circuitry. A control unit including hardware may also perform one or more of the techniques of this disclosure.

Such hardware, software, and firmware may be implemented within the same device or within separate devices to support the various operations and functions described in this disclosure. In addition, any of the described units, circuits or components may be implemented together or separately as discrete but interoperable logic devices. Depiction of different features as circuits or units is intended to highlight different functional aspects and does not necessarily imply that such circuits or units must be realized by separate hardware or software components. Rather, functionality associated with one or more circuits or units may be performed by separate hardware or software components or integrated within common or separate hardware or software components.

The techniques described in this disclosure may also be embodied or encoded in a computer-readable medium, such as a computer-readable storage medium, containing instructions that may be described as non-transitory media. Instructions embedded or encoded in a computer-readable storage medium may cause a programmable processor, or other processor, to perform the method, e.g., when the instructions are executed. Computer readable storage media may include random access memory (RAM), read only memory (ROM), programmable read only memory (PROM), erasable programmable read only memory (EPROM), electronically erasable programmable read only memory (EEPROM), flash memory, a hard disk, a CD-ROM, a floppy disk, a cassette, magnetic media, optical media, or other computer readable media.

Various examples have been described. These and other examples are within the scope of the following claims.

What is claimed is:

1. A medical device system comprising:
a handheld patient remote, the handheld patient programming remote having a configurable single actuation mechanism; and
processing circuitry configured to:
during a programming phase of a medical device, receive an indication of a type of the medical device to be in communication with the handheld patient programming remote;
during the programming phase, receive user input indicative of a user preference;
based on the indication of the type of medical device and the user preference, determine a function to be performed with the single actuation mechanism of the handheld patient programming remote; and
configure the single actuation mechanism to perform, in response to patient action, the determined operation.

2. The medical device system of claim 1, wherein the handheld patient programming remote is configured to perform the determined function in response to one of:
a button press, wherein the handheld patient programming remote comprises a single button; or
the handheld patient programming remote being within a threshold distance from the medical device.

3. The medical device system of claim 1, wherein the function to be performed with the single actuation mechanism of the handheld patient programming remote comprises one of:
a wake up function;
a scheduled therapy enablement and disablement function; or
a medical device power on and off function.

4. The medical device system of claim 1, wherein the single actuation mechanism comprises a button, and wherein, during a therapy phase, the handheld patient programming remote is configured to:
detect the medical device;
wirelessly connect to the medical device;
determine a status of the medical device;
determine whether the medical device is one of:
on or off,
wherein if the medical device is on, the handheld patient programming remote is configured to switch the medical device from on to off in response to a first button press of the button, and
wherein if the medical device is off, the handheld patient programming remote is configured to switch the medical device from off to on in response to a second button press of the button; or
enabled or disabled,
wherein if therapy of the medical device is enabled, the handheld patient programming remote is configured to switch the therapy of the medical device from enabled to disabled in response to a first button press of the button, and
wherein if the therapy of the medical device is disabled, the handheld patient programming remote is configured to switch the therapy of the medical device from disabled to enabled in response to a second button press of the button.

5. The medical device system of claim 1, wherein the single actuation mechanism comprises a button, and wherein during a therapy phase, the handheld patient programming remote is configured to:
receive an indication of a first button press of the button;
transmit a proximal signal; and
wirelessly connect to the medical device.

6. The medical device system of claim 1, wherein the single actuation mechanism comprises a button, and wherein during a therapy phase, the handheld patient programming remote is configured to:
receive an indication of a first button press of the button;
transmit a proximal signal; and
switch to an idle state in response to one of:
a threshold period of time passing; or
receiving an indication of a second button press.

7. The medical device system of claim 1, further comprising:
a patient electronic device, wherein the patient electronic device is configured to adjust one or more therapy configurations of the medical device.

8. The medical device system of claim 1, wherein the handheld patient programming remote comprises one or more light emitting diodes (LEDs).

9. The medical device system of claim 1, wherein the medical device comprises one of:
a tibial neuromodulation device;
a pudendal neuromodulation device;
a deep brain stimulation device;
a drug pump;
a spinal cord stimulation device; or
a sacral neuromodulation device.

10. The medical device system of claim 1, wherein the processing circuitry of the system comprises processing circuitry of a clinician programming device of the system.

11. A method comprising:
receiving, by processing circuitry of a medical device system and during a programming phase of a medical device, an indication of a type of the medical device to be in communication with a handheld patient programming remote, the handheld patient programming remote having a configurable single actuation mechanism;
receiving, by the processing circuitry and during the programming phase, user input indicative of a user preference;
determining, by the processing circuitry and based on the indication of the type of medical device and the user preference, a function to be performed with the single actuation mechanism of the handheld patient programming remote; and
configuring, by the processing circuitry, the single actuation mechanism to perform, in response to patient action, the determined operation.

12. The method of claim 11, wherein the handheld patient programming remote is configured to perform the determined function in response to one of:
receiving an indication of a button press, wherein the handheld patient programming remote comprises a single button; or
determining the handheld patient programming remote is within a threshold distance from the medical device.

13. The method of claim 11, wherein the function to be performed with the single actuation mechanism of the handheld patient programming remote comprises one of:
a wake up function;
a scheduled therapy enablement and disablement function; or
an medical device power on and off function.

14. The method of claim 11, wherein the single actuation mechanism comprises a button, the method further comprising:
detecting, by the handheld patient programming remote and during a therapy phase, the medical device;
wirelessly connecting, by the handheld patient programming remote and during the therapy phase, to the medical device;
determining, by the handheld patient programming remote and during the therapy phase, a status of the medical device;
determining, by the handheld patient programming remote and during the therapy phase, whether the medical device is one of:
on or off,
wherein if the medical device is on, the handheld patient programming remote is configured to switch the medical device from on to off in response to a first button press of the button, and
wherein if the medical device is off, the handheld patient programming remote is configured to switch the medical device from off to on in response to a second button press off the button; or
enabled or disabled,
wherein if therapy of the medical device is enabled, the handheld patient programming remote is configured to switch the therapy of the medical device from enabled to disabled in response to a first button press of the button, and
wherein if the therapy of the medical device is disabled, the handheld patient programming remote is configured to switch the therapy of the medical device from disabled to enabled in response to a second button press of the button.

15. The method of claim 11, wherein the single actuation mechanism comprises a button, the method further comprising:
receiving, by the handheld patient programming remote and during a therapy phase, an indication of a first button press of the button;
transmitting, by the handheld patient programming remote and during the therapy phase, a proximal signal; and
wirelessly connecting, by the handheld patient programming remote and during the therapy phase, to the medical device.

16. The method of claim 11, wherein the single actuation mechanism comprises a button, the method further comprising:
receiving, by the handheld patient programming remote and during a therapy phase, an indication of a first button press of the button;
transmitting, by the handheld patient programming remote and during the therapy phase, a proximal signal; and
switching, by the handheld patient programming remote and during the therapy phase, to an idle state in response to one of:
a threshold period of time passing; or
receiving an indication of a second button press.

17. The method of claim 11, wherein the medical device system further comprises:
a patient electronic device, wherein the patient electronic device is configured to adjust one or more therapy configurations of the medical device.

18. The method of claim 11, wherein the handheld patient programming remote comprises one or more light emitting diodes (LEDs).

19. The method of claim 11, wherein the medical device comprises one of:
a tibial neuromodulation device;
a pudendal neuromodulation device;
a deep brain stimulation device;
a drug pump;
a spinal cord stimulation device; or
a sacral neuromodulation device.

20. A non-transitory computer-readable medium storing instructions that when executed cause processing circuitry to:
during a programming phase of a medical device, receive an indication of a type of the medical device to be in communication with a handheld patient programming remote, the handheld patient programming remote having a configurable single actuation mechanism;
during the programming phase, receive user input indicative of a user preference;
based on the indication of the type of medical device and the user preference, determine a function to be performed with the single actuation mechanism of the handheld patient programming remote; and
configure the single actuation mechanism to perform, in response to patient action, the determined operation.
